(19) United States
(12) Patent Application Publication
(10) Pub. No.: US 2011/0034868 A1
(43) Pub. Date: Feb. 10, 2011

(54) SYSTEM FOR INJECTING A FLUID THROUGH OR INTO HUMAN SKIN

(75) Inventors: Peter Eichhorst, Hohen Neuendorf (DE); Michael Trample, Fuerstenwalde (DE)

Correspondence Address: SUGHRUE MION, PLLC, 2100 PENNSYLVANIA AVENUE, N.W., SUITE 800, WASHINGTON, DC, 20037, US

(73) Assignee: PRIMOJEX GMBH, HOHEN NEUENDORF (DE)

(21) Appl. No.: 12/866,198

(22) PCT Filed: Jan. 29, 2009
(86) PCT No.: PCT/EP2009/051014

§ 371 Date: Oct. 22, 2010

(30) Foreign Application Priority Data
Feb. 4, 2008 (DE) 10 2008 008 023.3

(51) Int. Cl. A61M 5/30; A61M 5/50

(52) U.S. Cl. 604/68; 604/110

(57) ABSTRACT

The invention relates to a system for injecting a fluid through or into human skin, optionally by needle-free injection or by injection with a needle, comprising
(A) a single-use syringe (10) comprising an ampule body (12) forming a chamber for accommodating the fluid to be injected, arranged within the chamber, an axially displaceable, sealingly guided ampule piston (16) and an external taper (18) of a Luer cone at the distal end of the ampule body (12);
(B) an adaptor (30) comprising an internal taper (32) of a Luer cone at the proximal end of the adaptor (30) and an exit aperture (36) for the fluid to be injected needle-free at the distal end of the adaptor (30) and
(C) an autoinjector comprising a receptacle device (50) for the coupled and fluid-filled combination of a single-use syringe (10) and an adaptor (30), which is configured to maintain the form-fit between the single-use syringe (10) and the adaptor (30) and to support the ampule body (12) of the single-use syringe (10), distally arranged a skin coupler (52) which forms a skin contact surface (56) for needle-free injection through or into human skin, wherein the skin coupler (52) is rounded and has a centrally arranged nozzle opening (54) which is configured to be complementary to the exit aperture (36) of the adaptor (30) and accommodates the exit aperture (36) of the adaptor (30) flush with the nozzle opening (54) in the region of the skin contact surface (56) during needle-free injection, and an injection device which is configured to displace the ampule piston (16) axially according to a predetermined pressure profile.

The invention relates to a system for injecting a fluid through or into human skin, wherein the injection can optionally be performed without or with a needle. The invention also relates to components configured, in particular, for the system according to the invention, specifically a single-use syringe, an adaptor and an autoinjector.

Technological background and prior art Needle-free injection devices for injecting a fluid—which usually contains medication—through or into human skin are known in a variety of forms from the prior art. The known devices have it in common that the fluid is ejected through a very small exit aperture of an ampule with application of a high pressure, wherein the fluid reaches a very high exit velocity sufficient to pass through the skin or penetrate into the skin and thus for needle-free subcutaneous or intradermal injection. Injection devices of this type comprise an ampule with an ampule body forming a chamber for accommodating the fluid to be injected. Arranged within the chamber is an axially displaceable, sealingly guided ampule piston. Also provided at the distal end of the ampule is an exit aperture for the fluid to be injected. The region of the ampule around said exit aperture defines, with proper use, the available skin contact surface for needle-free injection through or into human skin.

Relatively recent technical developments in this field attempt to combine needle-free injection with the conventional injection method using a conventional needle. Both systems are intended to be matched to one another such that use can be made of standardized conventional products. A high level of compatibility minimizes injection costs and leads to a high degree of market acceptance. A combination of this type does away with the logistical effort for the provision of both injection systems at the point of use and the material, manufacturing and storage costs can be significantly reduced.

DE 103 40 613 A1 discloses a device which is usable for injecting a fluid in the form of a single-use syringe or—by removing certain components of the device at defined predetermined breaking points—as pre-filled ampules for a needle-free injector. The device is therefore initially configured for the conventional injection route via a needle and can be converted to needle-free use. However, the predetermined breaking points bring with them the danger of an unwanted breakage during needle injection. The predetermined breaking points are also not sufficiently smooth in order to meet the requirements of needle-free injection in all respects.

U.S. Pat. No. 5,769,138 describes an adaptor with a spike cap for a needle-free injector. For this purpose, the adaptor and the injector each comprise coupling elements which serve to fix the adaptor on the injector. Additional sealing elements on the adaptor are intended to prevent the entry of air into the chamber of the injector. U.S. Pat. No. 5,919,159 also describes an adaptor with a spike attachment for a needle-free injector.

DE 10 2007 008 597.6 describes an injection system for injecting a fluid through or into human skin, which enables needle-free injection to be performed by means of conventional single-use syringes which are actually designed only for needle injection. In this case, an injection adaptor and an autoinjector are used. The adaptor is configured so that it can be connected to conventional single-use syringes and simultaneously comprises the skin contact surface with the injection nozzle for needle-free injection. The autoinjector accommodates the single-use syringe and can apply a predetermined pressure profile to the injection fluid.

This system is suitable, in particular, for single-use syringes that are not pre-filled. On manual filling of the single-use syringe, the adaptor must be removed from the syringe before filling. This poses the problem, in particular, of air bubbles which form, depending on syringe construction, after manual filling of single-use syringes and subsequent placement of the adaptor, and have to be removed subsequently under visual monitoring. The interface of the skin with the injection nozzle is conceived as part of the adaptor which is usually constructed as a disposable item. This leads to high costs for the single-use injection articles. It also requires a high level of attention by the injecting individual to ensure the sterility of the adaptor during removal and subsequent replacement during filling.

There is therefore a need for an improved injection system for needle-free injection or alternatively with an injection needle, wherein it is possible to resort to single-use syringes, convenient and air bubble-free manual filling is possible, and sterility is maintained during injection preparation. The costs of the single-use articles is also to be markedly reduced.

### Inventive Solution

The problem is preferably solved with a system for injection of a fluid through or into human skin, optionally by needle-free injection or by injection with a needle, having the features of claim 1. The inventive system comprises
(A) a single-use syringe comprising an ampule body forming a chamber for accommodating the fluid to be injected, arranged within the chamber, an axially displaceable, sealingly guided ampule piston and an external taper of a Luer cone at the distal end of the ampule body;
(B) an adaptor comprising an internal taper of a Luer cone at the proximal end of the adaptor and an exit aperture for the fluid to be injected needle-free at the distal end of the adaptor and
(C) an autoinjector comprising a receptacle device for the coupled, and fluid-filled, combination of single-use syringe and adaptor, which is configured to maintain the form-fit between the single-use syringe and the adaptor and to support the ampule body of the single-use syringe, arranged distally a skin coupler which forms a skin contact surface for needle-free injection through or into human skin, wherein the skin coupler is rounded and has a centrally arranged nozzle opening which is configured to be complementary to the exit aperture of the adaptor and accommodates the exit aperture of the adaptor flush with the nozzle opening in the region of skin contact during needle-free injection, and an injection device which is configured to displace the ampule piston according to a pre-definable pressure profile.

The system according to the invention therefore consists of three components, specifically a single-use syringe, an adaptor and an autoinjector. The components are matched to one another to enable injection optionally with a needle or needle-free. The single-use syringe was optimized for the requirements of needle-free injection. In the following, it is only needle-free injection that will be considered, since the use of the single-use syringe with a conventional needle attachment is generally known and therefore requires no detailed explanation.

Where the invention concerns the penetration of human skin, this should be understood to mean the penetration of fluid at least through the epidermis and preferably also through the corium of the skin.

The single-use syringe can correspond, in the geometry and materials thereof, to a conventional single-use syringe as used for needle injection. It comprises an ampule body with a chamber in which the fluid to be injected is stored. At the distal end of the ampule body is an interface which is configured, for needle-free injection, for the sealing and reversible securing of an attachment, specifically the adaptor according to the invention. The interface is configured as a Luer cone.

At the proximal end, the ampule body has an aperture in which an ampule piston lies axially displaceable and sealingly guided. The ampule piston can be configured as a hollow body or a solid body and can therefore have an increased stability for compression and bending compared with conventional piston rods for a needle-free injection procedure with an autoinjector. The piston rod is usually widened at the proximal end and this partial region of the ampule piston serves as a thumb pressure surface when properly used during needle injection.

At the distal end, the cylindrical piston rod has a tip which tapers distally, the contour of which is configured complementary to the dead volume in the interior of the external taper of the Luer cone. The piston rod therefore tapers to a tip and entirely fills the interior space of the external taper with this tip. A piston ring which seals relative to the ampule wall can preferably be mounted at the beginning of the taper of the piston rod, and this also serves during the injection as a stop and prevents the piston rod being pushed out of the ampule piston. The piston rod and the tip are preferably configured integral.

The design of the single-use syringe according to the invention makes it possible for the single-use syringe to be filled air bubble-free. In particular, the extension of the piston rod to a tip, so that the single-use tip has minimal dead volume, prevents the formation of air bubbles during the filling procedure. Such a design of the single-use syringe is not known to the applicant from the prior art and therefore represents a further claimed aspect of the invention.

A further aspect of the invention concerns an adaptor which is configured for interaction with the system according to the invention. The adaptor has a proximal internal taper of a Luer cone at the proximal end of the adaptor and an exit aperture for the fluid to be injected needle-free at the distal end of the adaptor. This means that the exit aperture for the fluid to be injected and the connection which is complementary to the interface of the single-use syringe are situated on the opposing sides of the adaptor.

The adaptor is preferably configured at the distal end such that a protective cap which protects the injection nozzle against unintended contamination during placement of the adaptor onto the single-use syringe can be placed on said adaptor. The protective cap can therefore be configured as a sterile cap and can be made from the plastics materials that are commonly used in medical technology.

For manual filling of the single-use syringe, the protective cap is removed. The adaptor remains on the single-use syringe during the filling procedure. The syringe tip is thus additionally extended so that the removal of fluids from break-open ampules is facilitated. If an additional adaptor or cannula is necessary for removing the fluid from the supply container, due to the second standard interface in the form of the distal external taper, said additional adaptor or cannula can easily be mounted on the adaptor. Following the filling procedure, the sterile cap is replaced on the adaptor and ensures the sterility of the system until utilization in the autoinjector.

When the single-use syringe according to the invention is used with a conventional injection needle, the adaptor can easily be removed from the single-use syringe together with the protective cap.

The adaptor preferably has, at the proximal end thereof, a peripheral groove and the receptacle device of the autoinjector has a corresponding fixing element which is configured to engage, in the closed state of the autoinjector, in the groove of the adaptor. By this means, fixing of the adaptor in the autoinjector can be easily realized.

The adaptor is preferably made from surgical stainless steel, so that it has a high degree of mechanical stability. Surgical stainless steel is also a biocompatible material that is often used in medical technology. The geometric configuration of the adaptor is kept simple and makes use of standard connections normally used in medical technology. In advantageous manner, this enables existing machines and knowledge of existing production methods to be used. Compared with the needle-free injection systems known from the prior art, this leads to a marked reduction in production costs.

A further aspect of the invention concerns an autoinjector, which is configured for interaction with the system according to the invention. The autoinjector comprises a receptacle device which is configured for receiving a combination, filled with the fluid, of a single-use syringe and an adaptor such that a form fit can be maintained between the single-use syringe and the adaptor and an ampule body of the single-use syringe is supported, a distally arranged skin coupler which forms a skin contact surface for needle-free injection through or into human skin, wherein the skin coupler is rounded and has a nozzle opening centrally, and an injection device which is configured to displace an ampule deflecting piston of the single-use syringe axially according to a pre-settable pressure profile. The receptacle device and the injection device are accommodated in a common housing. The skin coupler which accommodates the injection nozzle and is placed on the skin during needle-free injection is fastened to the receptacle device.

The receptacle device serves to accommodate the coupled combination, filled with the fluid, of the single-use syringe and the adaptor. Said receptacle device comprises an upper shell and a lower shell, wherein the upper shell is configured pivotable transversely to the longitudinal axis of the autoinjector and is displaceable in the longitudinal direction of the autoinjector. The lower shell is used for direct insertion of the single-use syringe with the adaptor. The receptacle device is also defined in the geometry thereof such that a form-fit is maintained between the single-use syringe and the adaptor, which means that the seal of the system during needle-free injection is ensured. The receptacle device also supports the ampule body of the single-use syringe. The latter measure serves essentially to prevent bursting of the ampule body on application of pressure and to reduce or prevent potentially elastic behavior of the ampule body on application of pressure and any consequent undesirable influence on the pressure profile of the emerging fluid. Therefore, the ampule body preferably lies against complementary support elements of the receptacle device of the autoinjector over the whole region surrounding the chamber.

The injection device of the autoinjector serves to pre-define a particular pressure profile of the emerging fluid as needed for needle-free injection. For this purpose, the ampule piston must be axially displaced accordingly in the ampule body of the single-use syringe. Both the pressure profile striven for and the means necessary in principle for implementation can be found in the needle-free injectors of the prior art. These means comprise, in general, an actuator, which is in engagement with the piston rod and the movement of which causes the stroke of the piston rod in the ampule body. An injection device of this type can be implemented entirely mechanically or with electronic control of the movement sequence. Of importance for the inventive purpose is only that the desired pressure profile can be predetermined for needle-free injection. For the actual design of the autoinjector, a person skilled in the art would be able to resort accordingly to the wealth of forms and the technical solutions of conventional needle-free injectors.

In conventional systems for needle-free injection, when injection pressure is applied to the fluid, the elastic behavior of the piston plug made from an elastic material must first be compensated for. As a consequence of the design of the ampule piston of the single-use syringe according to the invention, no elastic behavior of the system, which would otherwise lead to damping of the pressure rise during penetration, is observable. There is therefore no need for the formation of a complex pressure profile to compensate for this damping. Advantageously, due to the design according to the invention, the injection pressure profile can be built up directly in the single-use syringe (i.e., a high pressure impulse to penetrate the dermis and to form an injection channel, followed by a low dispersion pressure pattern for administering the intended remaining injection dose into the target tissue).

The skin coupler according to the invention comprises a rounded half shell which is fastened to the upper shell of the autoinjector. This rounded region forms a skin contact surface for needle-free injection through or into human skin. For interpretation of the expression “rounded,” reference is made to the disclosure of DE 10 2004 007 257 A1; particularly notable are the sectional views of the ampule bodies of FIGS. 4 to 6 and the associated part of the description of the drawings and the disclosure made in paragraph 6 of the publication. DE 10 2004 007 257 A1 describes a needle-free injection device of the aforementioned type, wherein the exterior configuration of the distal surface forming the skin contact surface of the ampule body has a convex and essentially edge-free contour. A contour of this type is described herein as being rounded.

Arranged centrally in the skin coupler is a nozzle opening which accommodates the distal exit aperture of the adaptor during needle-free injection. The nozzle opening is therefore geometrically complementary to the outer wall of the distal exit aperture of the adaptor. The nozzle opening and the exit aperture of the adaptor together form the injection aperture of the system. The skin coupler is preferably made from a biocompatible metal, such as surgical stainless steel.

Preferably, the skin contact surface is altered, for example, coated so as to have a greater power of adhesion to human skin compared with the untreated part of the skin coupler. This is intended to prevent slipping of the autoinjector filled with the adaptor and the single-use syringe on the skin, once applied. In particular, for this purpose, a coating is used which is applied concentrically around the nozzle opening of the skin coupler and is made from a biocompatible elastic material. The elastic material preferably consists of rubber, particularly butyl rubber, or a silicone-containing material.

The skin coupler is fastened to the upper shell of the receptacle device, wherein the fastening preferably takes place via at least one spring element. In particular, two spring elements are used. The spring element(s) preferably has/have, overall, one spring constant in the range of 10 N/m to 30 N/m. The spring elements are also selected such that, in the loaded rest condition, the skin coupler extends beyond the adaptor. This position serves a temporary protective function to preserve the sterile conditions for the adaptor between removal of the protective cap after insertion in the autoinjector until coupling on the skin immediately before needle-free injection.

For needle-free injection, the skin coupler must be pressed against the injection site. Due to the fastening according to the invention by means of spring elements, the skin coupler is displaced axially in the direction of the autoinjector. This has the result that the distal end of the adaptor with the injection aperture is positioned in the nozzle opening. In a preferred embodiment, the spring constant is precisely large enough so that at least the application force necessary for needle-free injection is achieved when the injection nozzle has reached the end position thereof in the nozzle opening. Preferably the system according to the invention is configured such that triggering of the mechanism for applying the pressure profile to the injection fluid only becomes possible when the injection nozzle has reached the end position thereof in the nozzle opening. A predetermined injection profile can only be achieved with needle-free injection when the injection takes place with a controlled and defined minimum application force on the skin. This is advantageously ensured with the design of the fastening of the skin coupler according to the invention. The system according to the invention for needle-free injection is therefore particularly suitable for autoinjection of a patient.

### BRIEF DESCRIPTION OF THE DRAWINGS

The invention will now be described in greater detail making reference to an exemplary embodiment and the associated drawings, in which:

FIG. 1 shows a single-use syringe, an adaptor and a protective cap for the system according to the invention;

FIG. 2 shows a schematic sectional view through the skin coupler, the adaptor and the distal end of the single-use syringe in the rest position following loading and during injection;

FIG. 3 shows a schematic exploded view to illustrate a receptacle device of the autoinjector in conjunction with the insertion of a coupled combination of adaptor and filled single-use syringe, and

FIG. 4 shows schematically the progression of a pressure profile that can be realized with the aid of an injection device of the autoinjector.

### DETAILED DESCRIPTION OF THE INVENTION

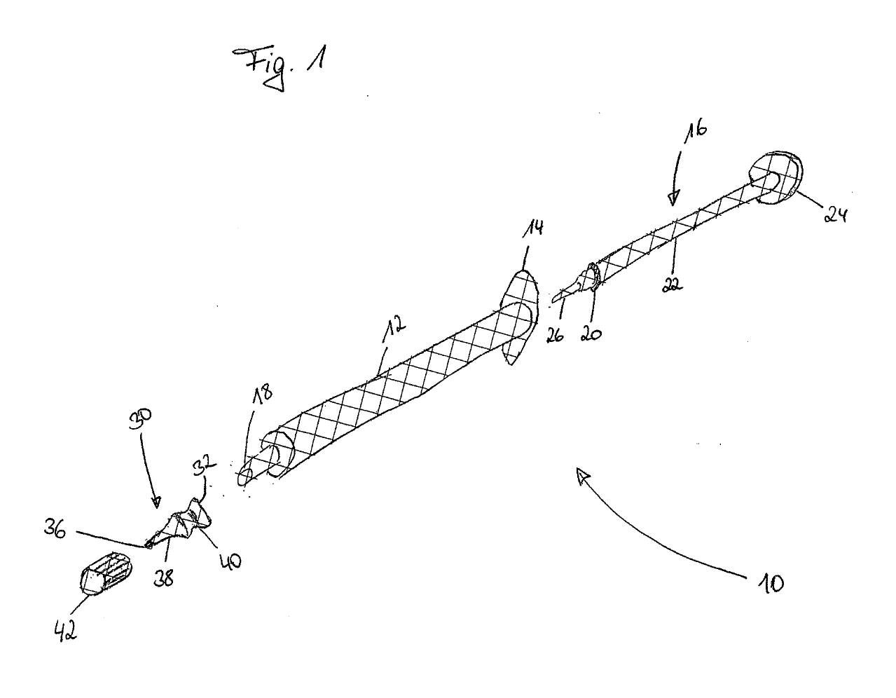

FIG. 1 shows a single-use syringe 10, an adaptor 30 and a protective cap 42 of the system according to the invention for injection, optionally with a needle or for needle-free injection of a fluid into human skin.

The single-use syringe 10 comprises a conventionally designed ampule body 12, the internal space of which consists of a chamber suitable for accommodating the fluid to be injected. An outer taper 18 of a Luer cone adjoins the ampule body 12 distally. Proximally, the ampule body 12 is extended to a stop 14 and is open. In the assembled condition of the single-use syringe 10, arranged in the chamber provided by the ampule body 12 is an ampule piston 16 which is guided axially displaceable and sealingly therein. The ampule body 16 also contains a cylindrical piston rod 22 which tapers distally to a tip 26. The tip 26 is formed so as to be geometrically complementary to the internal space of the external taper 18 and thus the dead space volume of the external taper 18 is completely filled in the pushed-in condition. At the start of the taper of the tip 26, a piston ring 20 which serves as a seal and a stop is placed on the cylindrical piston rod 22. The proximal end of the cylindrical piston rod 22 is widened to improve operability and represents a thumb pressure plate 24 for a conventional injection by means of injection needle. Both the cylindrical piston rod 22 and the ampule body 12 are made from a conventional plastics material used in medical technology, for example, polypropylene.

Compared with conventional ampule pistons, the ampule piston 16 shown in FIG. 1 is modified in the region of the cylindrical piston rod 22. The cylindrical piston rod 22 is essentially adapted to the dimensions of the chamber of the ampule body 12. The cylindrical piston rod 22 can, in particular, be implemented from the material used as a solid body or a profile body. This counteracts the risk of bending and breaking which exists as a consequence of the high pressure application necessary with needle-free injection.

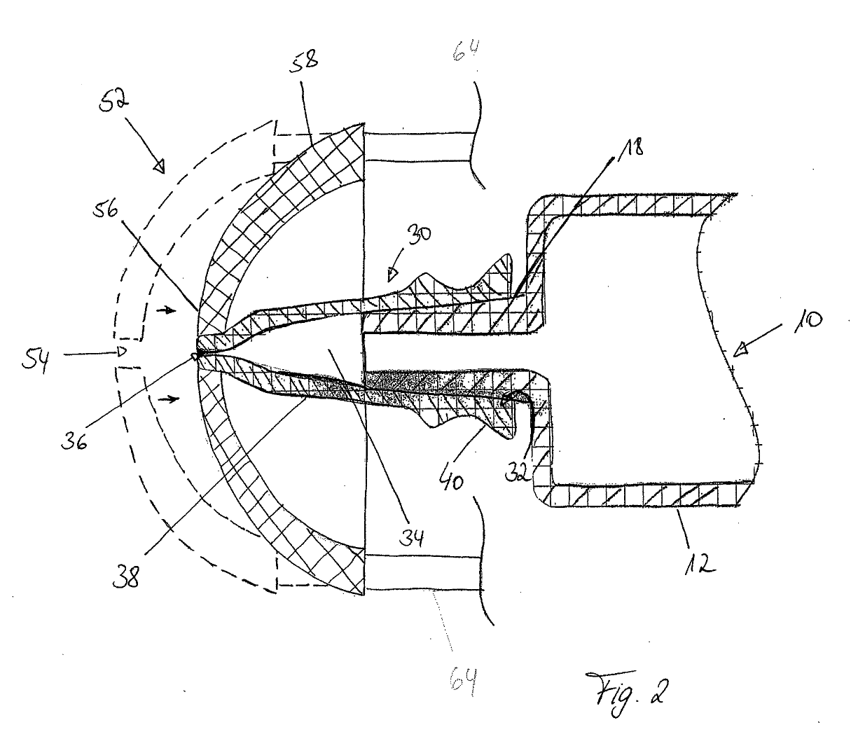

The adaptor 30 is made from surgical stainless steel. The structure of the adaptor 30 will now be described in greater detail making reference to the sectional representation in FIG. 2. The protective cap 42 can be made from a conventional plastics material used in medical technology, for example, polypropylene. The protective cap 42 has an internal taper which is configured complementary to the distal region of the adaptor 30 or, more precisely, to a distal external taper 38. The internal taper of the protective cap 42 is configured as a female Luer cone.

FIG. 2 shows a schematic sectional view through a skin coupler 52 as part of an autoinjector and the adaptor 30 after placement onto the single-use syringe 10, specifically both after loading (dashed edges) and during an injection. The section shown runs along a longitudinal axis of the combination consisting of single-use syringe 10, adaptor 30 and skin coupler 52, wherein only the distal end of the ampule body 12 of the single-use syringe 10 is shown.

As can be seen, the adaptor 30 is placed on the external taper 18 of the single-use syringe 10, that is the male Luer cone. For this purpose, the adaptor 30 has a complementary proximal internal taper 32, that is a female Luer cone, ensuring a force-fitting, sealing hold of the adaptor 30 on the single-use syringe 10.

The proximal internal taper 32 is adjoined by a channel 34 which is accessible to the fluid to be injected and which opens in a distally arranged exit aperture 36. The distal region of the adaptor 30 tapers continually toward the exit aperture 36. The adaptor 30 also has an external taper 38 of a Luer cone. On the outer side of the proximal region, the adaptor 30 also has a peripheral groove 40, which serves for fixing the adaptor 30 in the autoinjector.

The skin coupler 52 has a central nozzle opening 54, which accommodates the exit aperture 36 of the adaptor 30 during injection. The skin coupler 52 with the nozzle opening 54 and the exit aperture 36 of the adapter 30 together form a skin contact surface 56. During injection, the distal external taper 38 of the adaptor 30 ends flush with the surface of the skin coupler 52 in the region of the exit aperture 36.

The skin contact surface 56 has an additional coating 58 which is applied concentrically around the nozzle opening 54 of the skin coupler 52. The coating 58 serves to improve the slip-proofing on proper use, that is when placing the skin contact surface 56 on human skin. The material of the coating 58 is configured so that it has a better adhesive power in relation to human skin than the smooth hard skin contact surface 56.

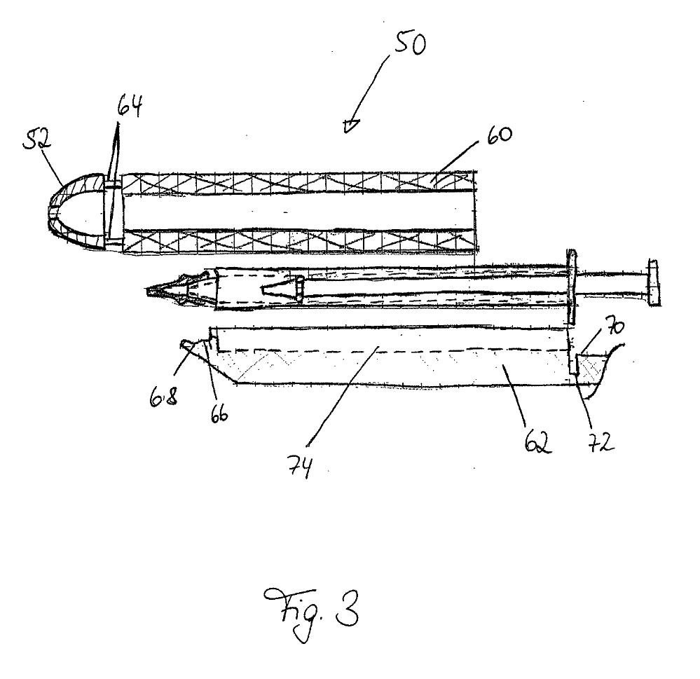

FIG. 3 shows, in a schematic exploded view, part of the autoinjector used for the system according to the invention, specifically the receptacle device 50 thereof with the skin coupler 52. The receptacle device 50 of the autoinjector is configured in two parts and comprises an upper shell 60 and a lower shell 62. In order to open the autoinjector and insert the combination of adaptor 30 and filled single-use syringe 10 into the lower shell 62, the upper shell 60 can be pivoted transversely to the longitudinal axis of the autoinjector and displaced in the longitudinal direction (not shown in detail here). The two parts of the receptacle device 50 can have largely the same contour in order to facilitate the production of the autoinjector. A configuration of this type is shown in FIG. 3, wherein the two components each have a hemicylindrical basic form. The skin coupler 52 is connected via spring elements 64 to the autoinjector. The spring elements 64 for fastening the skin coupler 52 are mounted only on the upper shell 60.

In the rest condition before the injection with the spring elements 64 relaxed, the skin coupler 52 is far enough removed from the single-use syringe 10 that the exit aperture 36 of the adaptor 30 is set back in the rear region of the skin coupler 52 (see FIG. 2). In order to change from the rest condition into the injection condition, the spring elements 64 on the receptacle device 50 on the autoinjector must be compressed.

Arranged at the distal end of the receptacle device 50 is a lug 66 with an adjacent groove 68, the geometry of which is matched by the peripheral groove 40 and the end region of the adaptor 30. Proximally, the receptacle device 50 has a further lug 70 with a further adjacent groove 72 which are configured in their dimensions such that they are able to accommodate the stop 14 of the ampule body 12. Extending between the two grooves 68, 72 is a hemicylindrically formed support region 74.

When the combination of the adaptor 30 and the single-use syringe 10 is inserted, these elements lie in form-fitting manner together, that is, the adaptor 30 is fixed on the single-use syringe 10 and can no longer be axially displaced relative thereto. This ensures the seal of the system during needle-free injection. In the closed condition, the hemicylindrical support region 74 lies against the ampule body 12. The whole ampule body 12 is supported by the hemicylindrical support region 74 in the region of the chamber, so that the significantly increased pressures that are usually to be expected with needle-free injection as compared with injection with a needle cannot not lead to bursting or expansion of the ampule body 12.

The autoinjector also comprises an injection device—not shown here—which is configured to displace the ampule piston 16 axially according to a predetermined pressure profile. An actuator which is configured in conventional manner engages on the ampule piston 16 and displaces said piston in a defined manner in the direction of the adaptor 30. The means for realizing the desired movement are sufficiently well known from the prior art, so that a detailed description is not required. It is significant for the purpose of the invention only that the pressure profile required for needle-free injection is produced.

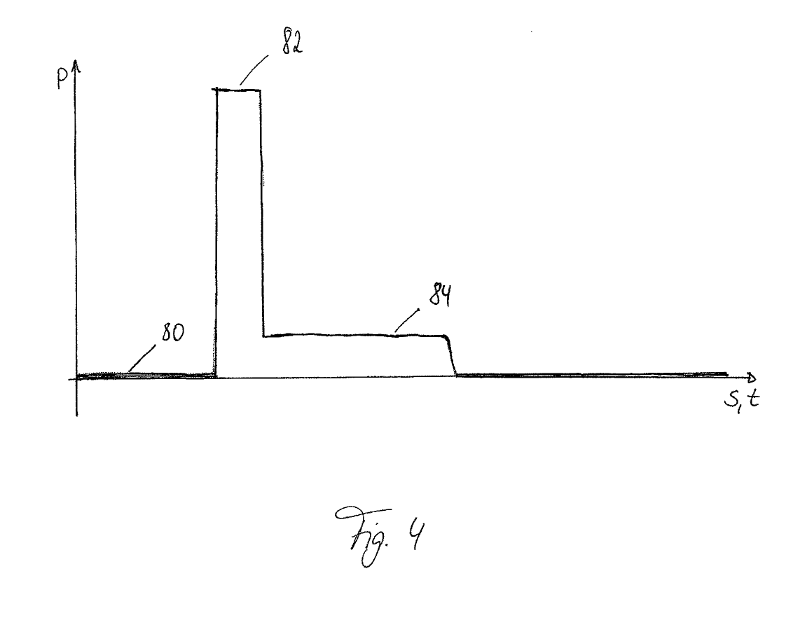

The pressure profile is shown schematically in FIG. 4 for illustration purposes. The low pressure that is otherwise usual at the start of a needle-free injection to compensate for the elastic behavior of the piston plug of the single-use syringe 10 is not required. The pressure profile initially shows a basic level 80 which is directly adjoined by an initial pressure peak 82. The initial pressure peak 82 has a high pressure and a small volume and serves to generate an injection channel into the tissue. The pressure then falls to an actual injection pressure 84, which means the pressure that is necessary for the actual injection of the remainder of the fluid, and the therapeutically required quantity of fluid is injected.

### REFERENCE SIGNS

10 Single-use syringe
12 Ampule body
14 Stop
16 Ampule piston
18 External taper
20 Piston ring
22 Cylindrical piston rod
24 Thumb pressure plate
26 Tip
30 Adaptor
32 Internal taper
34 Channel
36 Exit aperture
38 Distal external taper
40 Peripheral groove
42 Protective cap
50 Receptacle device
52 Skin coupler
54 Nozzle opening
56 Skin contact surface
58 Coating
60 Upper shell
62 Lower shell
64 Spring element
66, 70 Lug
68, 72 Groove
74 Hemicylindrical support region
80 Base level
82 Pressure peak
84 Injection pressure

1. System for injection of a fluid through or into human skin, by needle-free injection or by injection with a needle, comprising:
(A) a single-use syringe (10) comprising
an ampule body (12) forming a chamber for receiving the fluid to be injected;
an axially displaceable, sealingly guided ampule piston (16) arranged in the chamber; and
an external taper (18) of a Luer cone at the distal end of the ampule body (12);
(B) an adaptor (30) comprising
an internal taper (32) of a Luer cone at the proximal end of the adaptor (30);
at the distal end of the adaptor (30), an exit aperture (36) for the fluid to be injected needle-free; and
(C) an autoinjector comprising
a receptacle device (50) for the coupled and fluid-filled combination of a single-use syringe (10) and an adaptor (30), which is configured to maintain the form-fit between the single-use syringe (10) and the adaptor (30) and to support the ampule body (12) of the single-use syringe (10);
a distally arranged skin coupler (52) which forms a skin contact surface (56) for needle-free injection through or into human skin, wherein the skin coupler (52) is rounded and has a centrally arranged nozzle opening (54) which is configured to be complementary to the exit aperture (36) of the adaptor (30) and accommodates the exit aperture (36) of the adaptor (30) flush with the nozzle opening (54) in the region of the skin contact surface (56) during needle-free injection; and
an injection device which is configured to displace the ampule piston (16) axially according to a predetermined pressure profile.

2. Single-use syringe (10) for a system for injection of a fluid through or into human skin, by needle-free injection or by injection with a needle, comprising: an ampule body (12) forming a chamber for accommodating the fluid to be injected, arranged within the chamber, an axially displaceable, sealingly guided ampule piston (16) and an interface (18) of a Luer cone at the distal end of the ampule body (12),
wherein:
the ampule body (16) comprises a cylindrical piston rod (22) having a distally tapering tip (26), the contour of which is configured complementary to the dead volume in the interior of the external taper (18) of the Luer cone.

3. Adaptor (30) for a system for injection of a fluid through or into human skin, by needle-free injection or by injection with a needle, comprising: a proximal internal taper (32) of a Luer cone at the proximal end of the adaptor (30) and an exit aperture (36) for the fluid to be injected needle-free at the distal end of the adaptor (30).

4. The adaptor (30) as claimed in claim 3,
wherein
the adaptor (30) has at the distal end thereof a distal external taper (38) of a Luer cone.

5. The adaptor (30) as claimed in claim 3,
wherein
the adaptor (30) is made from surgical stainless steel.

6. Autoinjector for a system for injection of a fluid through or into human skin, by needle-free injection or by injection with a needle, comprising:
a receptacle device (50) which is configured for receiving a combination, filled with the fluid, of a single-use syringe (10) and an adaptor (30) such that a form fit is maintained between the single-use syringe (10) and the adaptor (30) and an ampule body (12) of the single-use syringe (10) is supported,
a distally arranged skin coupler (52) which forms a skin contact surface (56) for needle-free injection through or into human skin, wherein the skin coupler (52) is rounded and has a nozzle opening (54) centrally, and
an injection device which is configured to displace an ampule piston (16) of the single-use syringe (10) axially according to a predetermined pressure profile.

7. The autoinjector as claimed in claim 6,
wherein
the skin coupler (52) has a coating (58) on the skin contact surface (56), which prevents slipping of the skin coupler (52) on the skin and consists of an elastic, biocompatible material.

8. The autoinjector as claimed in claim 7,
wherein
the coating (58) of the skin contact surface (56) is butyl rubber and/or silicone.

9. The autoinjector as claimed in claim 6,
wherein
the receptacle device (50) comprises an upper shell (60) and a lower shell (62), wherein the upper shell (60) is configured pivotable transversely to the longitudinal axis of the autoinjector and is configured displaceable in the longitudinal direction of the autoinjector.

10. The autoinjector as claimed in claim 6,
wherein
the skin coupler (52) is fastened to the upper shell (60) of the receptacle device (50).

11. The autoinjector as claimed in claim 10,
wherein
the fastening of the skin coupler (52) to the upper shell (60) takes place via at least one spring element (64).

12. The autoinjector as claimed in claim 11,
wherein
the spring element or spring elements (64) have a spring constant overall in the range of 10 N/m to 30 N/m.

13. The adaptor (30) as claimed in claim 4,
wherein
the adaptor (30) is made from surgical stainless steel.
